(19) United States
(12) Patent Application Publication
(10) Pub. No.: US 2014/0011700 A1
(43) Pub. Date: Jan. 9, 2014

(54) THREE-DIMENSIONAL STRUCTURE OF H1N1 NUCLEOPROTEIN IN COMPLEX WITH ANTIVIRAL COMPOUNDS

(71) Applicants: Eric T. Baldwin, Upper Holland, PA (US); Patricia Ann McDonnell, Lumberville, PA (US); Suzanne Edavettal, San Diego, CA (US); Hal Lewis, Pennington, NJ (US); Bradley C. Pearce, East Hampton, CT (US); David R. Langley, Meriden, CT (US); Christopher W. Cianci, Madison, CT (US); Linda Discotto, New Britain, CT (US); Samuel Gerritz, Guilford, CT (US); Shuhao Shi, Madison, CT (US); Shirong Zhu, Cheshire, CT (US)

(72) Inventors: Eric T. Baldwin, Upper Holland, PA (US); Patricia Ann McDonnell, Lumberville, PA (US); Suzanne Edavettal, San Diego, CA (US); Hal Lewis, Pennington, NJ (US); Bradley C. Pearce, East Hampton, CT (US); David R. Langley, Meriden, CT (US); Christopher W. Cianci, Madison, CT (US); Linda Discotto, New Britain, CT (US); Samuel Gerritz, Guilford, CT (US); Shuhao Shi, Madison, CT (US); Shirong Zhu, Cheshire, CT (US)

(73) Assignee: Bristol-Myers Squibb Company

(21) Appl. No.: 14/003,003

(22) PCT Filed: Mar. 23, 2012
(86) PCT No.: PCT/US12/30221

§ 371 Date: Sep. 4, 2013

Related U.S. Application Data
(60) Provisional application No. 61/467,410, filed on Mar. 25, 2011.

(51) Int. Cl. C07K 14/005; G01N 33/68

(52) U.S. Cl. CPC C07K 14/005; G01N 33/6875
USPC 506/9; 436/501; 530/358

(57) ABSTRACT

The binding mode of the antiviral compounds have been characterized through a variety of biophysical and structural studies, elaborating on the proposed aggregation mechanism of action. We demonstrate the direct binding of these antiviral compounds to NP using thermal shift enhancement assay (TSE) and NMR. In addition, we have completed a detailed analysis of the oligomerization mechanism of action using dynamic light scattering, analytical ultracentrifugation, and surface plasmon resonance (SPR). Structure determination using x-ray crystallography confirmed the proposed compound-induced oligomerization mechanism of action. The co-crystal structure revealed that two compounds bound in an anti-parallel fashion bridging two NP monomers, inducing a novel non-native NP oligomer. Taken together, our data suggest a complex binding mode in which the compounds bind NP specifically in stoichiometric fashion inducing the formation of an NP oligomer without obstructing the RNA binding pocket or interfering with the native NP homo-oligomerization.

### FIELD OF THE INVENTION

The present invention relates generally to the three-dimensional structure of H1N1 nucleoprotein in complex with novel antiviral compounds. Additionally, the present invention relates to methods of designing and/or identifying H1N1 antiviral compounds. The present invention further relates to methods of modulating H1N1 nucleoprotein activity by defining key binding sites.

### INTRODUCTION

Influenza A viral infections result in annual epidemics of respiratory illness, with global death tolls ranging from 250,000 to 500,000. While annual vaccination is the primary defense against influenza infection, there is a clear need for both novel prophylactic and therapeutic antiviral therapies. Current antiviral therapies are targeted against the viral neuraminidase (zanamivir and oseltamivir) and the M2 ion channel protein (adamantanes). However, the propensity of Influenza A viruses to develop resistance to these antiviral compounds (Moscona, “Global transmission of oseltamivir-resistant influenza”, N. Engl. J. Med., 360(10):953-956 (2009)) (Regos and Bonhoeffer 2006) requires the development of novel antiviral therapeutics. (Regos and Bonhoeffer, “Emergence of Drug-Resistant Influenza Virus: Population Dynamical Considerations”, Science, 312:389-391 (2006)).

Influenza A is a single-stranded negative-sense RNA virus, with a genome consisting of 8 viral RNA (vRNA) segments encoding 11 proteins. The vRNA genome is encapsidated by nucleoprotein (NP), which serves to package the vRNA, providing higher order helical structure. Binding of the vRNA:NP complex to the vRNA polymerase forms the ribonucleoprotein (RNP) particle, a non-covalent circular complex that represents the functional template for vRNA replication. Formation of functional RNPs requires NP to interact directly with the vRNA, two of the vRNA polymerase subunits, PB1 and PB2, and to form NP:NP oligomers (Ruigrok et al., “Structure of influenza virus ribonucleoprotein particles. II. Purified RNA-free influenza virus ribonucleoprotein forms structures that are indistinguishable from the intact influenza virus ribonucleoprotein particles”, J. Gen. Virol., 76(Pt. 4):1009-1014 (1995); Elton et al., “Oligomerization of the influenza virus nucleoprotein: identification of positive and negative sequence elements”, Virology, 260(1):190-200 (1999); Elton et al., “Identification of amino acid residues of influenza virus nucleoprotein essential for RNA binding”, J. Virol., 73(9):7357-7367 (1999)). Viral replication can be effectively halted through molecular interference in any of the processes which result in NP binding to the vRNA or the viral RNA polymerase units, or the formation of NP oligomers (Newcomb et al., “Interaction of the influenza a virus nucleocapsid protein with the viral RNA polymerase potentiates unprimed viral RNA replication”, J. Virol., 83(1):29-36 (2009)) (Biswas et al., “Influenza virus nucleoprotein interacts with influenza virus polymerase proteins”, J. Virol., 72(7):5493-5501 (1998)) (Elton et al. 1999). In addition, NP also plays a critical role in nuclear import and export of RNPs and binds to a variety of host proteins (Portela et al., “The influenza virus nucleoprotein: a multifunctional RNA-binding protein pivotal to virus replication”, J. Gen. Virol., 83(Pt. 4):723-734 (2002)). Recent crystal structures of NP have spurred interest in this influenza protein as a potential target for vaccine design and antiviral development (Ng et al., “Structure of the influenza virus A H5N1 nucleoprotein: implications for RNA binding, oligomerization, and vaccine design”, FASEB J., 22(10):3638-3647 (2008); Ye et al., “The mechanism by which influenza A virus nucleoprotein forms oligomers and binds RNA”, Nature, 444(7122):1078-1082 (2006)). As such, NP has emerged as an attractive target for antiviral therapies.

A recent report by Kao et al (Kao et al., “Identification of influenza A nucleoprotein as an antiviral target”, Nat. Biotechnol., 28(6):600-605 (2010)) described a series of antiviral compounds which were identified using a cell-based Influenza A infection assay and subsequently demonstrated antiviral activity in vivo. These antiviral compounds were observed to inhibit NP nuclear transport. Kao, et al. proposed that inhibition of NP nuclear transport was due to compound induced formation of large NP complexes that aggregated RNA and possibly other cellular components. Herein we provide a detailed biophysical and structural analysis of the oligomerization mechanism of action for this novel series of antiviral compounds (FIG. 1). Direct binding of the compounds to NP was confirmed using thermal stability enhancement assay (TSE) and NMR experiments. Characterization of the NP:compound complexes using dynamic light scattering and analytical ultracentrifugation defined the size and binding stoichiometry of the complex. In addition, SPR analysis of RNA binding for the NP:compound oligomer demonstrated that compound-induced oligomerization of NP did not alter NP affinity for RNA. Structure determination using x-ray crystallography confirmed compound-induced oligomerization of NP. The observed complex consisted of two NP trimers, which retained native trimer interactions, cross-linked by 6 compound molecules resulting in a novel non-native NP oligomer.

### SUMMARY OF THE INVENTION

The present invention relates generally to the three-dimensional structure of H1N1 nucleoprotein in complex with novel antiviral compounds. Additionally, the present invention relates to methods of designing and/or identifying H1N1 antiviral compounds. The present invention further relates to methods of modulating H1N1 nucleoprotein activity by defining key binding sites.

One aspect of the invention defines a crystalline form, wherein the form is a co-crystal comprising H1N1 nucleoprotein and an antiviral compound selected from the group consisting of Compound A and analogs thereof.

Another aspect of the invention describes a H1N1 NP antiviral binding site comprising H1N1 NP monomers from different H1N1 NP trimers.

Another aspect of the invention is the H1N1 NP antiviral binding site, wherein the antiviral binding site comprises amino acids S376, E53, R99, S50, Y313, Y52, A284 of monomer B and Y289, Y52, A284, N309, R305 and Y289 of monomer A. More specifically, key amino acids of the NP antiviral binding site comprise Y52, Y289 and N309.

Yet another aspect of the invention is a method of identifying an H1N1 antiviral by providing the atomic coordinates of H1N1 NP polypeptide defining a three-dimensional structure of H1N1 NP polypeptide and then identifying chemical entities or fragments with the potential to bind to the antiviral binding site and synthesizing or acquiring the test compound; and determining the ability of the test compound to generate NP oligomers.

### BRIEF DESCRIPTION OF THE DRAWINGS

FIG. 1: Chemical structure of antiviral compounds. Compounds B-G are analogs of this core structure.

FIG. 2: The effect of several compounds on NP thermal stability. (A) Normalized TSE data for NP thermal denaturation alone (♦) and complexed with increasing concentrations of compound A (2.5 μM ▴, 5 μM X, 10 μM Δ, 25 μM ). (B) Thermal stability enhancement of a series of compounds correlates with influenza virus inhibition. The change in Tₘ, for 2 μM NP complexed with a series of inhibitors at a concentration of 25 μM. was plotted against EC₅₀ from a influenza virus tissue culture assay.

FIG. 3: Using NMR spectroscopy, the direct binding of compound A to NP was demonstrated. Downfield region of ¹H NMR spectra: (A) 48 μM compound A. (B) 48 μM compound A: 5 μM NP. (C) 48 μM compound A: 10 μM NP. (D) 48 μM compound A: 20 μM NP. (E) 48 μM compound A: 40 μM NP. (F) 20 μM NP.

FIG. 4: Dynamic light scattering characterization of NP:compound complexes. (A) Regularization histogram of 1 μM NP in 20 mM Tris pH 7.5, 1M NaCl, 5% glycerol, 8% DMSO. The autocorrelation function shown in (B) was used to calculate a hydrodynamic radius of 5.4 nm and polydispersity of 12.1%. The correlation function, describing the measured rate of time intensity fluctuations, is an exponential comprised of correlation coefficients (y-axis) dependent upon the delay time α-axis; the time-value separating the sets of data). The measured data (blue) is superimposed with the calculated autocorrelation function (purple). (C) The regularization histograms of NP (♦) and NP complexed with compounds A (▪), B (▾), C (▴) and D () indicate that the compounds are inducing the formation of higher-order oligomers. The regularization histogram for compound A alone (▴) demonstrates that compound A does not form large aggregates when solubilized under the reaction conditions.

FIG. 5: Characterization of the NP:compound complex by AUC. (A) SV-AUC protein detection (interference) data and fits for NP and the NP:compound complex. Analysis using interference, 280 nm, 360 nm reveals that formation of the NP:compound complex increases the rate of sedimentation. (B) SE-AUC data and fits for NP and the NP:compound complex. Equilibrium scans were globally fit with a monomer-Nmer model, and as expected indicate that NP is in equilibrium between monomer and trimer at 25C (average MW=121±6 kDa). At 280 nm, addition of an equimolar concentration of compound A results in a shift in oligomeric state to a mixture of monomer/trimer and trimer/octamer (average MW=304±6 kDa). Analysis of the data collected at 360 nm indicates that when NP is bound to compound A it exists in equilibrium of trimer/octamer (average MW=444±13.5 kDa

FIG. 6: SPR analysis of NP affinity for RNA. Sensorgram of the binding of NP to immobilized RNA was double referenced and fit to a 1:1 binding model. Data are shown as black lines with the fit in orange. The concentrations of NP injected are noted on the right side.

FIG. 7: Comparison of NP monomer structures. Variation in the tail loop domains is observed when NP monomers from the two published structures (PDB ID 21QH (green) and 2Q06 (purple)) were superimposed with the structure reported here (cyan).

FIG. 8: The structure of NP bound to compound A. (A) The NP:compound A complex contains two NP trimers cross-linked by six molecules of compound A. Each NP monomer is colored separately, with one trimer illustrated in shades of red and the other trimer illustrated in shades of blue. Compound A is bound between two molecules of NP at a flat interface on the body domain. The RNA binding pocket is unobstructed (denoted for two of the NP monomers). (B) Two inhibitors bind in an anti-parallel manner cross-linking NP monomers from different trimers, with each NP monomer contributing two half-compound binding sites. (C, D) The NP:compound complex as viewed from the side of the head domain of one of the NP trimers (trimer B and the compounds have been omitted for illustration purposes). The tail loop of one NP monomer binds to a cavity on the body domain of a neighboring monomer. Compound binding does not impact native tail-loop mediated oligomerization.

FIG. 9: NP residues involved in compound A binding. Compound A is colored in green, monomer A colored in red, and monomer B colored in blue. NP residues which form the compound binding site are illustrated as sticks and hydrogen bonds shown as dotted lines.

FIG. 10: Partial structure alignment of NP from four Influenza A viruses. A ClustalW alignment of NP from H3N2 (Brisbane/10/2007), H1N1 (A/Solomonlslands/3/2006), H1N1 (A/CA/07/2009), and A/WSN/33. Identical residues are colored yellow and homologous residues are colored blue. Known compound binding site mutations are denoted with red arrows and include Y52, E53, Y289, R305, N309, and Y313 (in A/WSN/33).

### DETAILED DESCRIPTION OF THE INVENTION

### Direct Binding of Compounds to NP

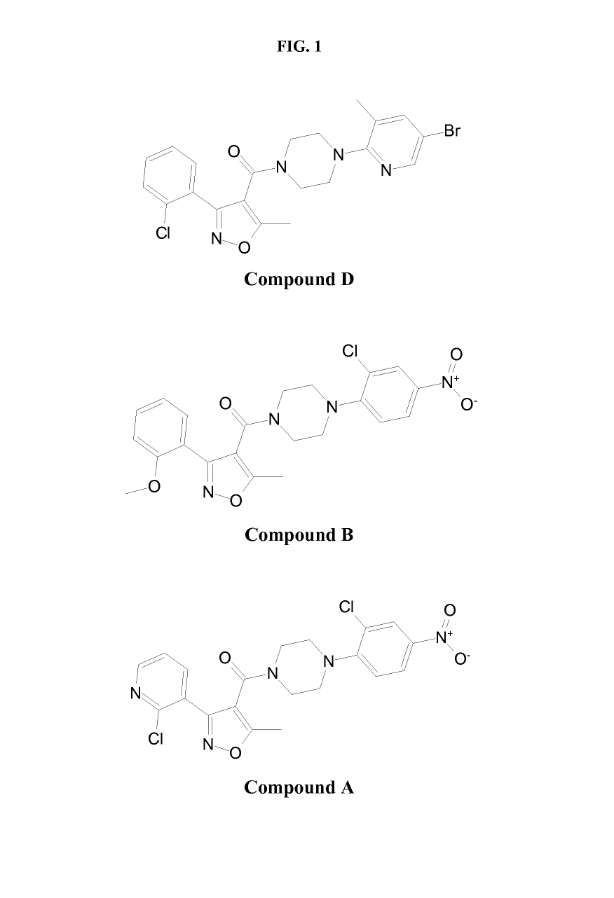

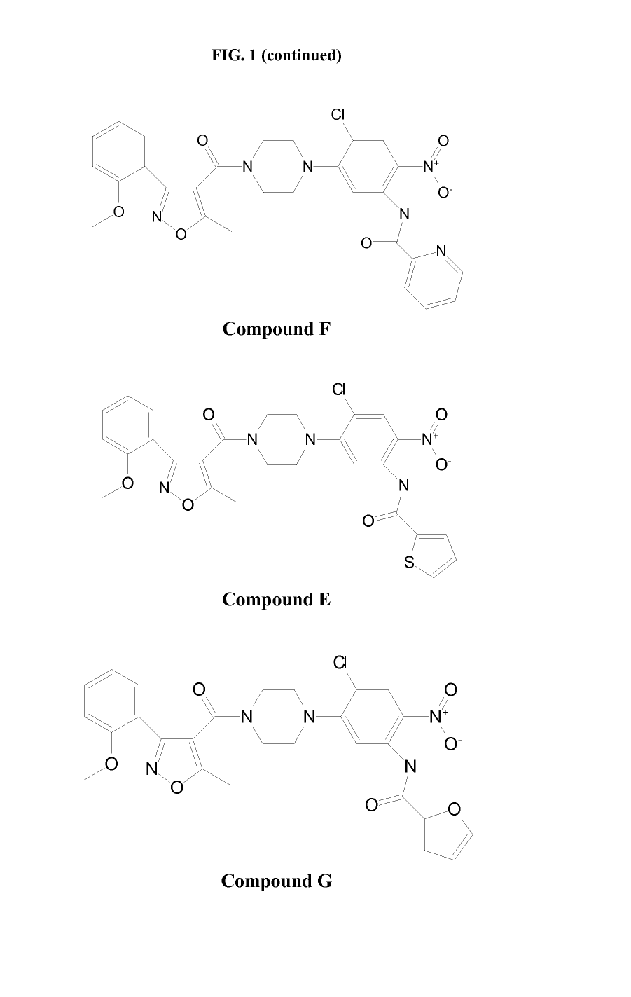

The antiviral compounds (FIG. 1) were confirmed to bind to NP using thermal stability enhancement. TSE compares the change in the unfolding transition temperature (ΔTₘ) of a protein with and without bound ligand. The TSE experiments described here used a fluorescent dye (ANS) to monitor the protein unfolding during thermal denaturation. As a protein unfolds and hydrophobic regions are exposed, an increase in ANS binding is observed as an increase in fluorescence intensity. The midpoint of the protein unfolding transition is defined as the Tₘ. A ligand that binds reversibly to the protein causes an increase in Tₘ that is proportional to both the ligand concentration and its binding affinity (Pantoliano et al., “High-density miniaturized thermal shift assays as a general strategy for drug discovery”, J. Biomol. Screen., 6(6):429-440 (2001); Lo et al., “Evaluation of fluorescence-based thermal shift assays for hit identification in drug discovery”, Anal. Biochem., 332(1):153-159 (2004); Cummings et al., “Universal screening methods and applications of ThermoFluor”, J. Biomol. Screen., 11(7):854-863 (2006); McDonnell et al., “Assessing compound binding to the Eg5 motor domain using a thermal shift assay”, Anal. Biochem., 392(1):59-69 (2009)).

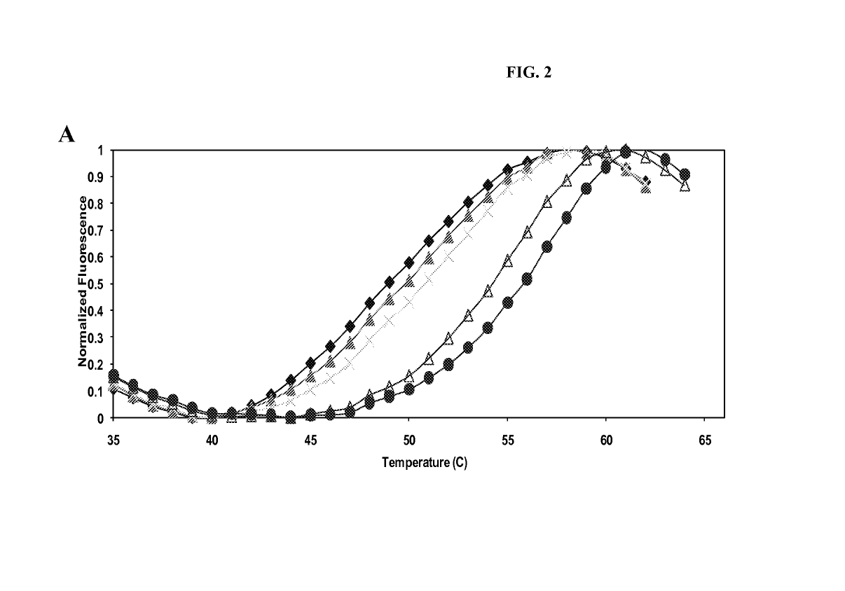

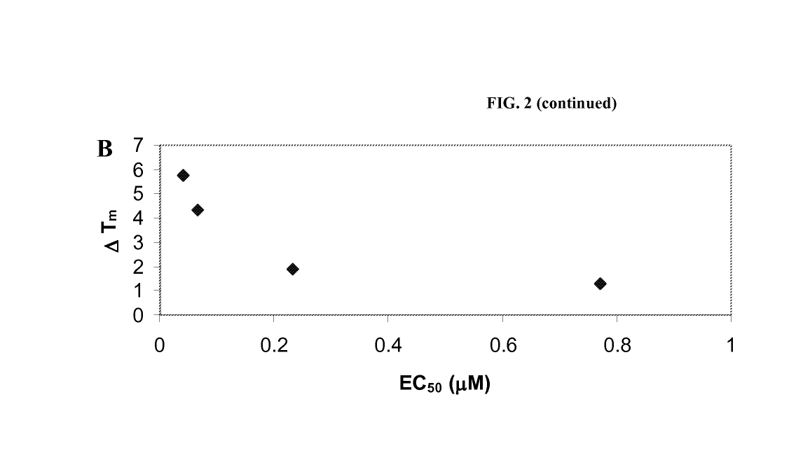

TSE conditions were optimized for NP and ANS concentrations, with the Tₘ for NP observed to be 51.3±0.2° C. The effect of several compounds on NP thermal stability was then evaluated. Concentration dependent thermal enhancement was observed for the compounds under study (FIG. 2A) suggesting a direct interaction of the compounds with NP. At the highest concentrations tested (25 μM) the Tₘ increased to 52.5-57.0° C. (ΔTₘ=1.2-5.7° C.) for this series of compounds, with the degree of thermal stability enhancement trending with compound potency as determined by the EC₅₀ for influenza virus inhibition (FIG. 2B).

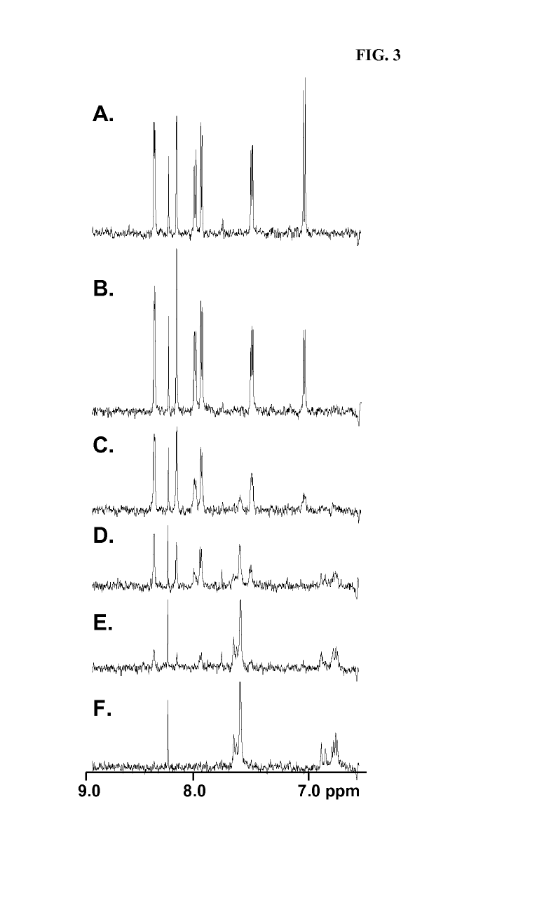

Direct binding of compounds A-D to NP was also confirmed using NMR T₂ relaxation experiments. The T₂ relaxation experiment exploits the difference in rotational correlation times between a small molecule and protein; binding of a small molecule to a large protein leads to a dramatic increase in effective molecular size of the small molecule, which is reflected as a decrease in its ¹H NMR signal in the T2-filtered NMR spectrum. Comparison of the downfield region of the ¹H NMR spectrum of compound A in the absence (FIG. 3A) and presence (FIG. 3B) of NP protein reveals the ¹H NMR signals of compound A are attenuated beyond detection upon the addition of an equimolar amount of NP. Similar data was observed for compounds B-D. These data confirm direct binding of this chemotype to NP.

### Biophysical Characterization of Compound-Induced NP Oligomerization

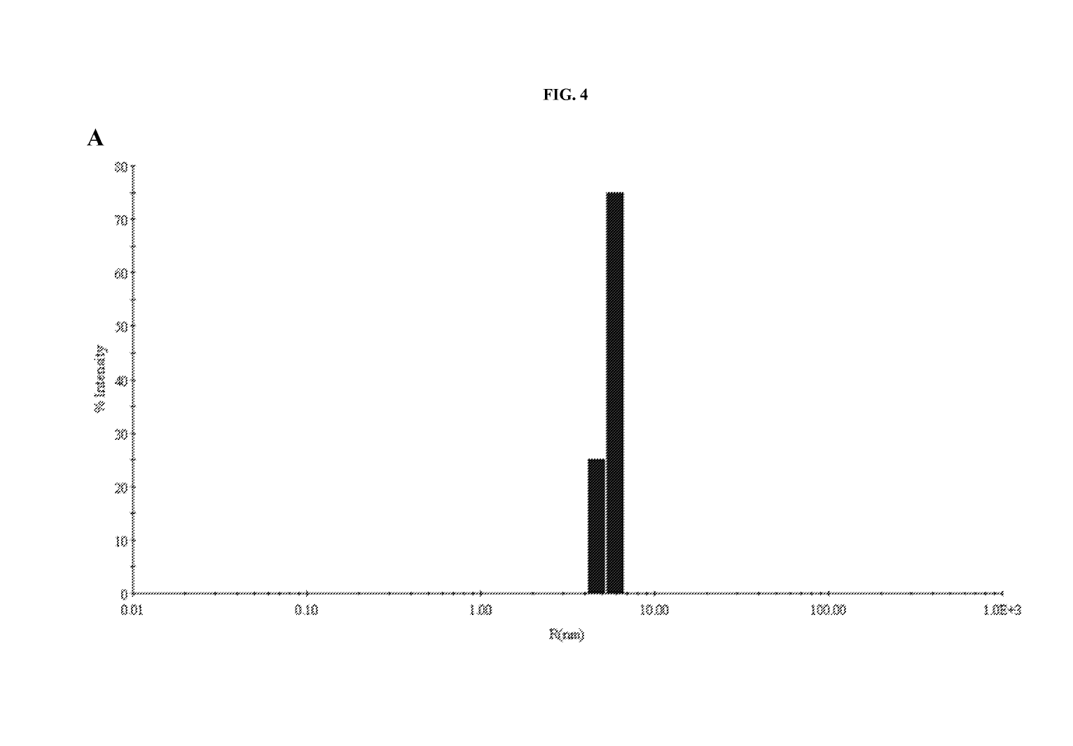

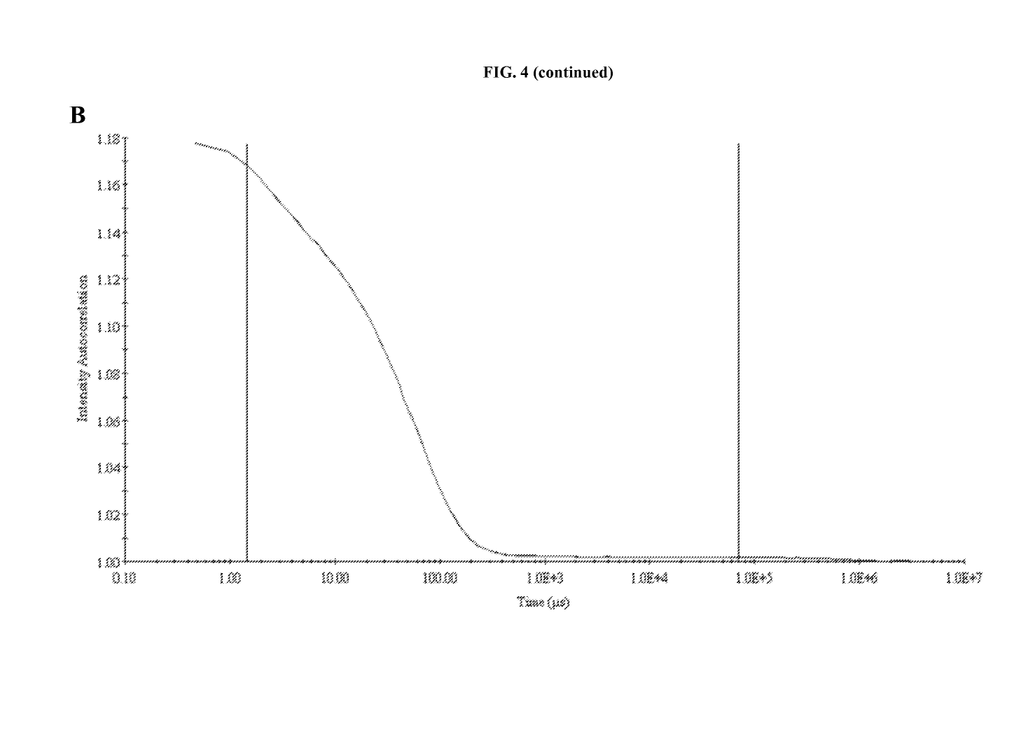

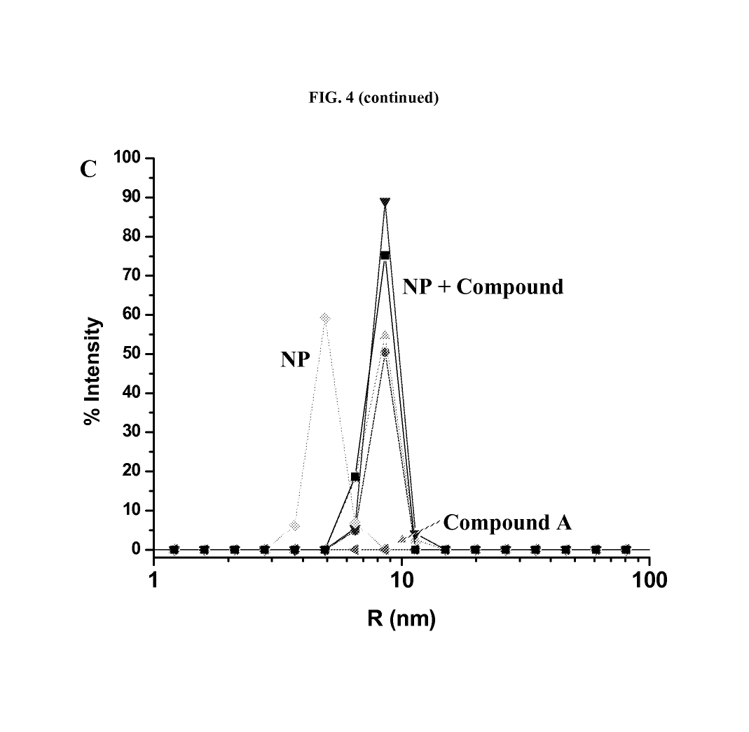

As NP oligomerization is known to be critical to its physiological function (Elton et al. 1999), the impact of compound binding on NP oligomerization was studied using dynamic light scattering (DLS). DLS provides a measure of particle size and size distribution in solution from the observed translational diffusion coefficient. DLS experiments were performed in triplicate, with the average and standard deviation reported here. Following regularization analysis using Dynamics V6 (Goldin, “Software for particle size distribution analysis in photon correlation spectroscopy” from www.softscientific.com/science/WhitePapers/dynals1/dynals100.htm), purified NP (1 μM) was found to have a hydrodynamic radius of 5.4±0.4 nm and was monodisperse (12.1% polydispersity) (FIG. 4A, 4B). The Raleigh spheres model was used to calculate MW-R, which is an estimate of the molecular weight interpolated from the hydrodynamic radius. The MW-R for NP was 175 kDa, which is consistent with SEC analysis and previous reports (Ye et al. 2006) that NP exists predominantly as a trimer in solution. Addition of compound (2-fold molar excess) results in an increase in the hydrodynamic radius to 8.1 nm, 8.2 nm, 8.5 nm, and 8.9 nm for compounds A-D as shown in Table 1 below and FIG. 4C.

TABLE 1
DLS Analysis of NP:Compound Complexes

| | Rₕ (nm) | % Pd |
|---|---|---|
| NP | 5.4 ± 0.4 | 12.1 |
| NP + Compound A | 8.1 ± 0.2 | 13.8 |
| NP + Compound B | 8.2 ± 0.3 | 10.2 |
| NP + Compound C | 8.5 ± 0.5 | 8.7 |
| NP + Compound D | 8.9 ± 0.2 | 13.5 |

Using the Raleigh spheres model, a hydrodynamic radius of 8.2 nm corresponds to a MW-R of 459 kDa. The formation of the complex did not dramatically alter the polydispersity, which remained between 8.7%-13.8%. These results suggest that the compound-induced oligomerization does not result in large heterogeneous or random aggregates, but rather a protein:compound complex of well-defined composition. Compounds were analyzed in the absence of NP and were not observed to form large aggregates when solubilized under these reaction conditions (data shown for compound A in FIG. 4C).

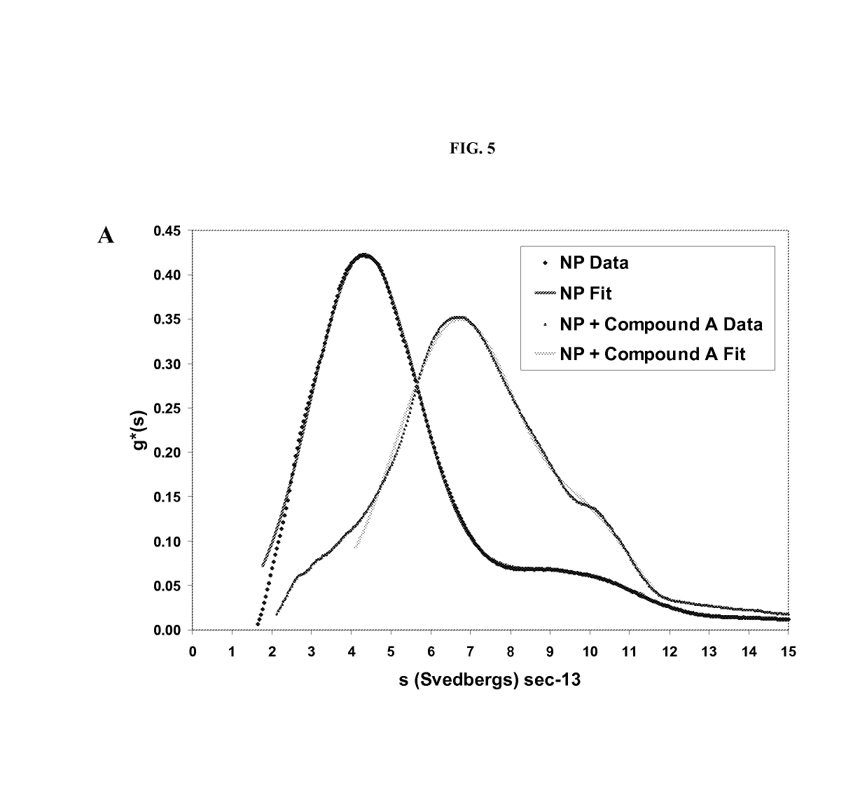

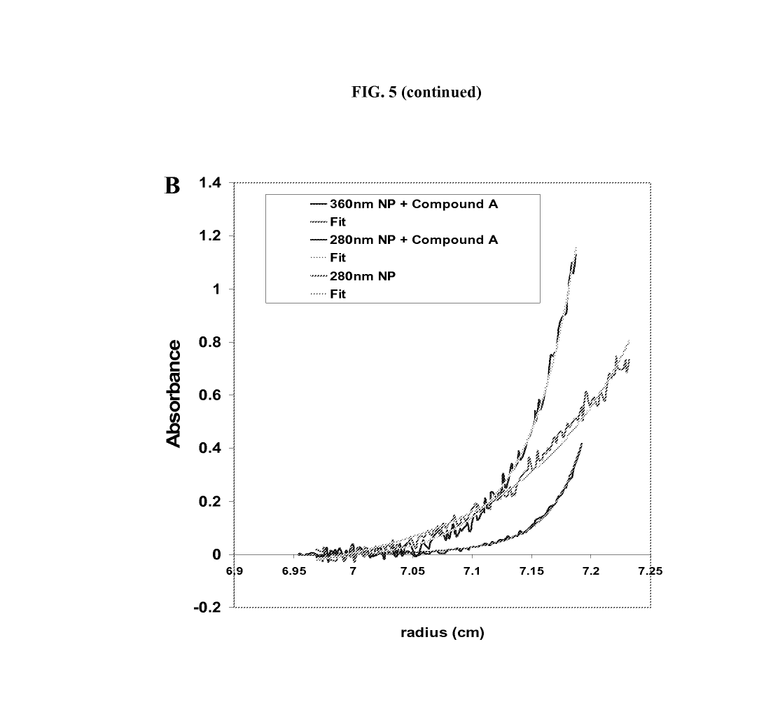

Analytical ultracentrifugation further characterized the observed compound-induced NP oligomerization. Compound A was chosen for these studies because it has a peak UV absorbance at 360 nm, allowing both the protein and compound sedimentation to be monitored by multiple wavelength scanning Sedimentation velocity experiments demonstrated that addition of compound A caused a shift in s-value from 4.5 to 7.2 as shown in FIG. 5A and Table 2 below.

TABLE 2
Sedimentation Velocity-AUC Analysis
of NP:Compound Complexes

| | Interference (S) | 280 nm (S) | 360 nm (S) |
|---|---|---|---|
| NP | 4.5, 9.5 | 4.5 | n.a. |
| NP + Compound A | 6.9, 10.2 | 7.2 | 7.1 |

Data collected at 360 nm demonstrated that the compound sediments with NP and the s-value calculated at this wavelength is the same as at 280 nm (7.1 S). These data indicate that inhibitor binding to NP increases the rate of sedimentation. Under these conditions (10 μM NP), a subpopulation of larger oligomers is observed (9.5 S); this oligomer shoulder appears to shift to 10.2 S in the presence of compound A. However this shoulder is broad, difficult to fit to a single s-value, and likely to contain a mixture of higher order oligomers. The shoulder in the absence and presence of compound largely overlaps and suggests that compound A does not significantly increase the oligomeric state of the larger oligomer.

Additional characterization of the compound-induced oligomerization of NP was completed using sedimentation equilibrium experiments. Equilibrium scans from multiple speeds were globally fit with a monomer-Nmer model using HeteroAnalysis (Cole, 2006, HeteroAnalysis, 1.133). In the absence of compound, NP was observed to be in a monomer-trimer equilibrium with an equivalence point of 9 μM at 25° C. (FIG. 5B). A single exponential fit to the NP data gave an average molecular weight of 121±6 kDa. The addition of an equimolar concentration of compound A shifted the observed average MW to 304±6 kDa, resulting in a shift in oligomeric state to a mixture of monomer/trimer and trimer/octamer. Analysis of the data collected at 360 nm for this same equimolar mixture indicated that when NP was bound to compound A it existed primarily as a higher order oligomer (average MW=444±13.5 kDa). Additional SE-AUC experiments were completed using a 2-fold molar excess of compound A. Under these conditions of excess compound A, the data at both 280 nm and 360 nm indicated that the formation of the NP:compound complex was shifted completely to higher oligomer (MW=432±14 kDa measured at 360 nm; MW=443±2 kDa measured at 280 nm). The binding stoichiometry was determined using the calculated extinction coefficient for NP at 280 nm, the measured extinction for compound A at 360 nm, and the area under the sedimentation curves. The respective concentrations of NP and compound A were determined to be 9.9±0.45 μM NP and 10.5±0.5 μM compound A, indicating a binding stoichiometry of 1 compound/monomer.

### Biacore Analysis of RNA Binding

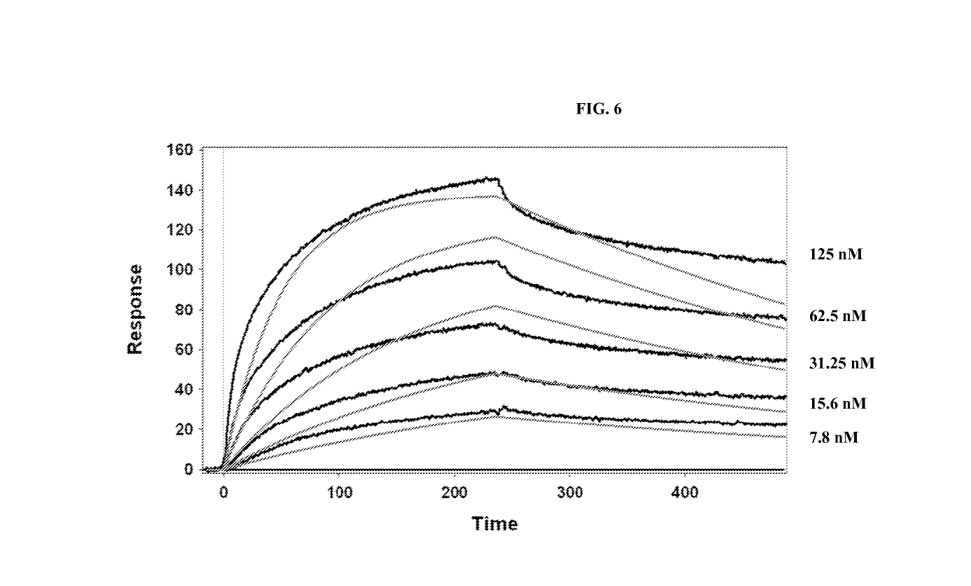

The impact of compound-induced oligomerization of NP on RNA binding affinity was evaluated by surface plasmon resonance (SPR). Based on structural and mutational studies NP is proposed to interact with RNA through an arginine-rich groove in a sequence nonspecific fashion (Albo et al., “Identification of an RNA binding region within the N-terminal third of the influenza A virus nucleoprotein”, J. Virol., 69(6):3799-3806 (1995); Elton et al. 1999). Trimeric NP has been proposed to interact with RNA first, which then results in NP assembly into higher-order oligomers and RNA encapsidation (Ng et al. 2008). Biotinylated RNA was immobilized on the surface of an SA chip. Varying concentrations of NP or NP:compound complex were flowed over the immobilized RNA (FIG. 6). The data were double referenced against either running buffer (NP analyte) or running buffer plus compound A (NP:compound complex analytes) and an unmodified surface, before fitting the data to a 1:1 binding model. The 1:1 binding model used for data fitting does not adequately describe the known complicated binding kinetics of NP for RNA (FIG. 6). Given the length of the RNA ligand used, each oligonucleotide is capable of binding two NP monomers and the possibility of NP oligomers cross-linking separate RNA ligands on the surface is likely. However, all data were fit using a simple model not as a means to accurately describe the binding kinetics but as the basis to compare RNA binding affinity for the different analytes. The measured RNA binding affinity of 11.2 nM for NP (Table 3 below) agrees well with previously published reports (Ng et al. 2008).

TABLE 3
SPR Analysis of NP:Inhibitor Complexes

| | K_{D} (nM) | kₐ (1/Ms) | k_{d} (1/s) | Rₘₐₓ |
|---|---|---|---|---|
| NP | 11.2 | 1.19E+05 | 2.20E−03 | 153.2 |
| NP + Compound A | 10.2 | 1.17E+05 | 1.09E−03 | 242.2 |
| NP N309K | 9.7 | 1.80E+05 | 1.73E−03 | 146.0 |
| NP N309K + Compound A | 11.5 | 1.75E+05 | 2.30E−03 | 144.1 |

NP complexed with a 2-fold molar excess of compound A was observed to have a similar binding affinity, 10.2 nM, as NP alone. However, the Rₘₐₓ value was greater for the NP:compound complex (242.2 RU) than for NP (153.2 RU), reflecting the increased size of the NP:compound complex. As a control, the same experiment was completed for NP containing a compound binding site mutation, N309K, which is known to greatly reduce NP affinity for compound A (Table 5 below).

TABLE 5
Comparison of the Potency of Compounds A-D Against
A/WSN/33 with Binding Site Mutations

| | | A/ | A/ | A/ | A/ |
|---|---|---|---|---|---|
| | A/ | WSN/33 | WSN/33 | WSN/33 | WSN/33 |
| | WSN/33 | Y52H | Y289H | N309T | N309K |
| | (EC₅₀, | (EC₅₀, | (EC₅₀, | (EC₅₀, | (EC₅₀, |
| | μM) | μM) | μM) | μM) | μM) |
| Compound | 0.07 ± 1.8 | n.a. | >20 | n.a. | >20 |
| A | | | | | |
| Compound | 0.04 ± 1.8 | 8.15 | >20 | 3.5 | >20 |
| B | | | | | |
| Compound | 0.27 ± 1.8 | >20 | >20 | >20 | >20 |
| C | | | | | |
| Compound | 1.02 ± 1.8 | >20 | >20 | >20 | >20 |
| D | | | | | |

The N309K mutation does not impact RNA binding affinity for NP with or without the addition of compound A (9.7 nM and 11.5 nM, respectively). The Rₘₐₓ values were similar for N309K with or without the addition of compound A (146 RU and 144.1 RU, respectively), reflecting the lack of compound binding and compound-induced oligomerization. Therefore, neither compound binding to NP nor compound-induced oligomerization of NP was observed to inhibit its ability to bind RNA.

### Structural Analysis of NP:Compound Complexes

A 3.2 Å structure of NP bound to compound A was determined The NP:compound structure reported here has the same space group and similar cell dimensions to the published H5N1 NP structure (Table 4 below) (Ng et al. 2008).

TABLE 4
Crystallization, Data Collection, and Refinement Statistics
(outer shell statistics in parentheses)

| | |
|---|---|
| Unit cell (Å, °) | 146.692 146.6925 146.692 |
| | 90.000 90.000 90.000 |
| Space group | P2₁3 |
| Resolution range (Å) | 44.23-3.00 |
| No. of reflections | 21313 (20480 working |
| | set, 833 test set) |
| No. of protein chains | 2 (A, B) |
| Ligand id codes | LG1 |
| No. of protein residues | 853 |
| No. of ligands | 2 |
| No. of waters | 7 |
| R_{work} | 0.2331 |
| R_{free} | 0.2959 |
| Rmsd bond lengths (Å) | 0.006 |
| Rmsd bond angles (°) | 0.915 |
| Number of disallowed φψ angles | 6 |

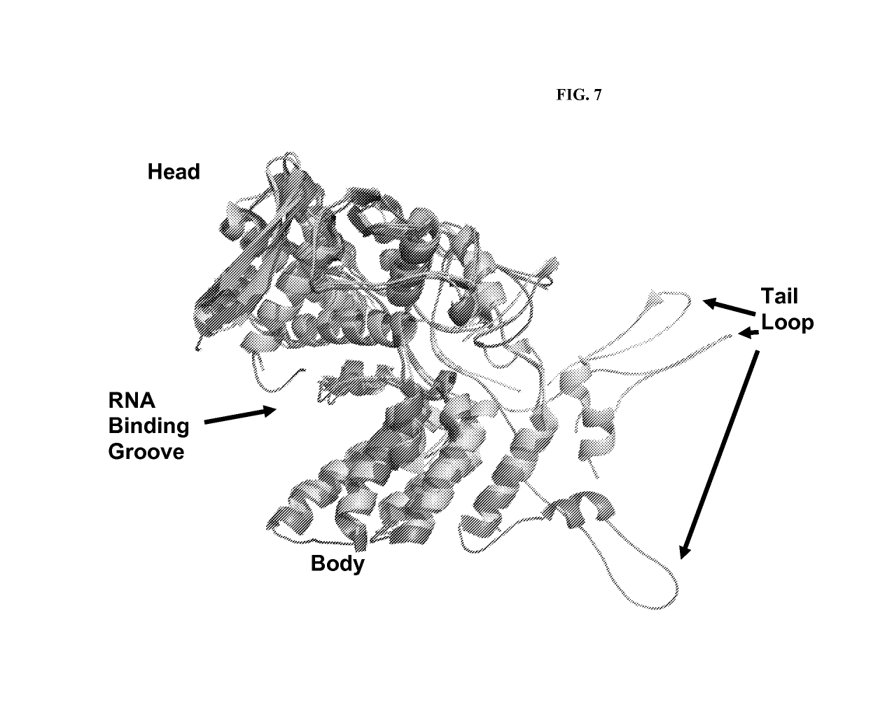

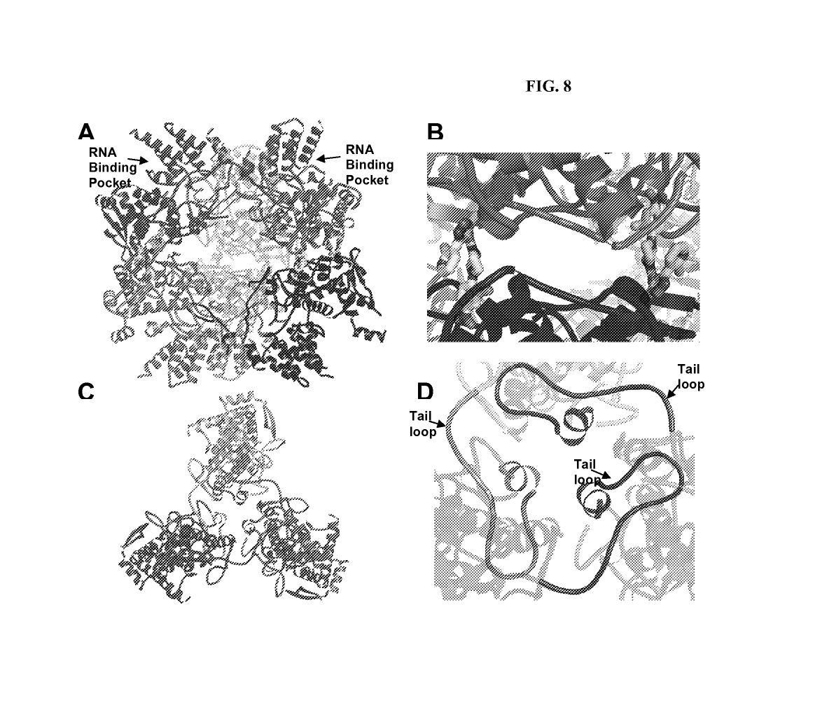

The overall fold of each NP monomer is similar to published structures, being predominately α-helical, having a crescent-like shape with head, body, and tail loop domains (FIG. 7). A comparison of this structure with the previously published NP structures reveals the flexibility of the tail loop domain, with different orientations observed in each structure. Tail-loop flexibility is likely critical to the numerous physiological functions of NP, most importantly the varying orientations of monomers within the RNP necessary for RNA encapsidation (Ng et al. 2008). The tail loop domain mediates NP oligomerization by inserting into a cleft in the body domain of an adjacent monomer to form an intertwined triad (FIG. 7, 8D) (Ye et al. 2006). As was observed in the H5N1 NP structure, this structure has two monomers in the asymmetric unit, each of which is part of a tail-loop mediated trimer about the crystallographic 3-fold axis (FIG. 8).

Two molecules of compound A bind in an anti-parallel manner at a flat interface on the body domain of NP, cross-linking NP monomers from different NP trimers (FIG. 8). Interestingly, compound binding does not impact tail-loop mediated oligomerization, rather induces a new non-native oligomer. This non-native oligomer consists of two NP trimers cross-linked by six molecules of compound A (FIG. 8C, D). The RNA binding pocket is unobstructed by the formation of the non-native oligomer, as was predicted by the SPR data that demonstrated compound binding does not alter RNA binding affinity (FIG. 8A).

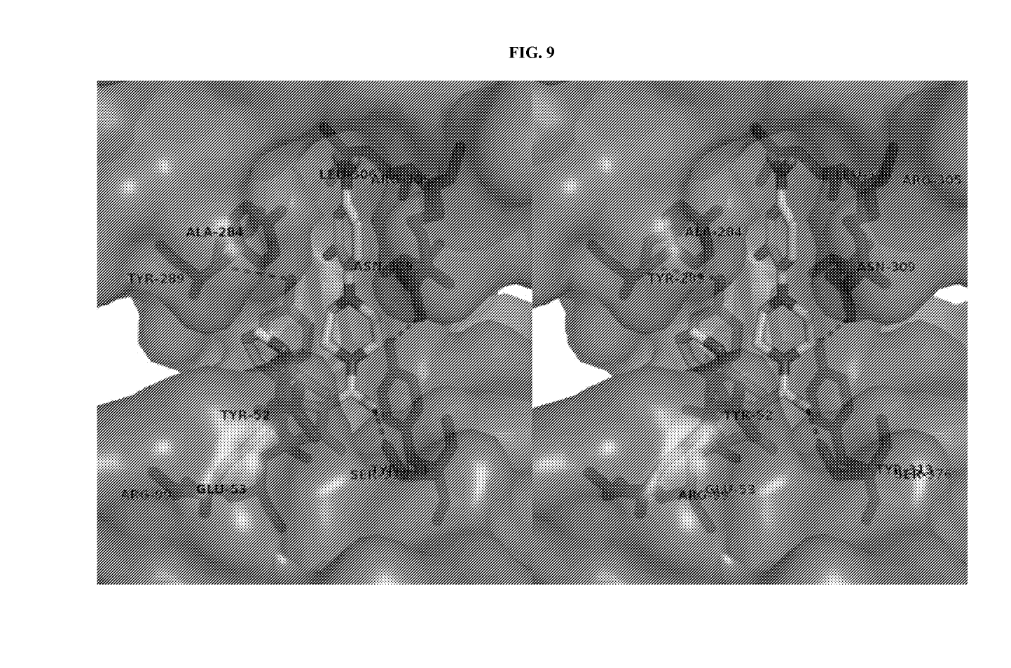

Key NP:compound interactions are mediated by 5376, E53, R99, R305, and Y289 (FIG. 9). S376 (monomer B) is observed to form a hydrogen bond to the piperazine carboxyl moiety of compound A. The E53 backbone amide (monomer B) forms a key hydrogen bond to S50 (monomer B) and R99 (monomer B) providing the structural pocket around the isoxazole binding site. R99 (monomer B) also provides π-cation interactions with isoxazole aryl ring. Packing interactions are observed between the nitro-aryl ring of the compound and the sidechains of R305 (monomer A) and Y289 (monomer A). Close inspection of the compound binding pocket reveals a hydrophobic wall formed by Y313 (monomer B), Y52 (monomer B), and Y289 (monomer A), which extends around the backside of the compound binding pocket and provides it stacking interactions with the compound's nitro-aryl ring. Interestingly, two novel NP:NP interactions are observed between compound cross-linked NP monomers. We observe hydrogen bonds between Y313 of monomer B and N309 of monomer A and between Y52 of monomer A and the backbone amide of A284 in monomer B. These interactions may play critical roles in stabilizing the non-native oligomer and thus contribute to the overall binding affinity of NP for compound A.

### Binding Pocket Variation

N309K, Y289H, and Y52H were identified from escape mutant viruses, which were resistant to the antiviral activity of this chemotype. Both N309K and Y289H were previously reported to be critical to compound binding (Kao et al. 2010). Activity analysis using these drug-selected A/WSN/33 viruses with NP containing N309K, Y289H, or Y52H mutations demonstrated that these residues are critical to compound potency, with a substantial reduction in antiviral activity observed (Table 5 above). Potencies for this chemotype varied from 40 nM for the WT A/WSN/33 to inactive for mutant viruses, demonstrating that these residues are critical to compound binding. The efficacy of this chemotype was also evaluated against three common Influenza A viruses, H3N2 (Brisbane/10/2007), H1N1 (A/Solomonlslands/3/2006), and novel swine-origin H1N1 (A/CA/07/2009) (Table 6 below).

TABLE 6
Comparison of Potency of Compounds A-D with
a Variety of Common Influenza A Viruses

| | | | | A/Solomon- |
|---|---|---|---|---|
| | | A/Brisbane/ | A/CA/ | Islands/ |
| | A/WSN/33 | 10/2007 | 07/2009 | 3/2006 |
| | (EC₅₀, μM) | (EC₅₀, μM) | (EC₅₀, μM) | (EC₅₀, μM) |
| Compound A | 0.07 ± 1.8 | >20 | >20 | 3.49 |
| Compound B | 0.04 ± 1.8 | 7.31 ± 1.8 | >20 | 0.66 ± 0.22 |
| Compound C | 0.27 ± 1.8 | 12.74 ± 5.08 | >20 | 2.58 ± 1.53 |
| Compound D | 1.02 ± 1.8 | >20 | >20 | 2.80 ± 0.53 |

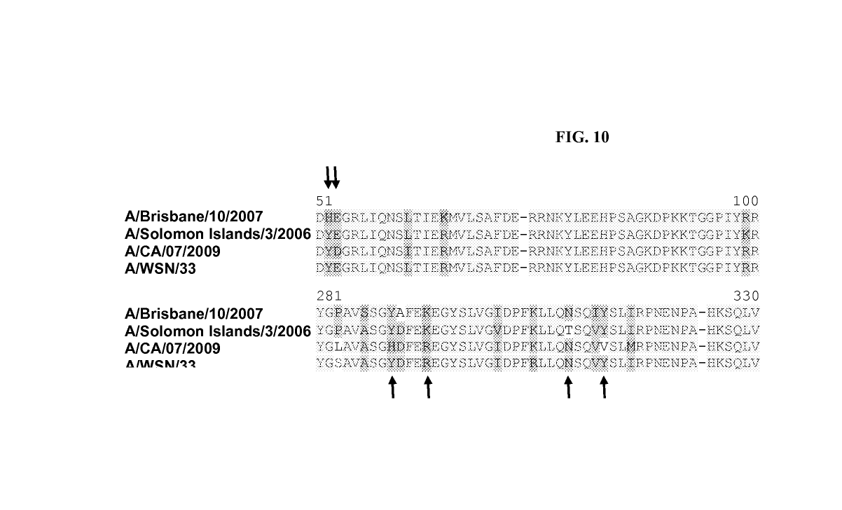

Compound potencies against these viruses varied from 40 nM to inactive, suggesting that the compound binding pocket is a region of some variability among NP from different Influenza A viruses. Sequence analysis of NP from these viruses revealed compound binding site mutations in common with the escape mutant viruses: A/CA/07/2009 contains an Y289H mutation, A/SolomonIslands/3/2006 contains a N309T, and A/Brisbane/10/2007 contains an Y52H mutation (FIG. 10). As described above, all three of these residues provide critical interactions for compound binding.

### Discussion

The tractability of a new antiviral influenza target, NP, was recently demonstrated for a series of compounds that have potent in vitro and in vivo antiviral activity (Kao et al. 2010). Kao et al., propose a mechanism of action by which the compounds trigger NP oligomerization, halt nuclear import of RNPs, and thereby inhibit viral replication. We have confirmed and elaborated on this novel mechanism, characterizing the binding mode through a variety of biophysical and structural studies. We confirmed that the antiviral potency of this series of compounds is due to direct binding to NP. DLS characterization the compound:NP complexes demonstrated that the compounds induce the formation of large, but well-defined NP oligomers. Based upon MW-R estimations, the DLS data suggested that the compounds induce formation of NP octamers, which was confirmed by AUC. These AUC studies also determined the binding stoichiometry to be 1 compound/monomer. As observed by SPR, NP:compound oligomer had similar binding affinity for RNA as NP alone, indicating that the formation of this large particle does not grossly alter the structure of NP or obstruct the RNA binding domain.

These biophysical data were confirmed using x-ray crystallography, with the structure demonstrating the compounds cross-link adjacent NP monomers from different NP trimers. Two compounds are bound in an anti-parallel orientation linking two NP monomers, without disruption of the native NP-NP tail loop mediated oligomerization. The 1:1 binding stoichiometry revealed structurally is in agreement with the calculated stoichiometry by AUC. In addition, the proposed RNA binding pocket is intact in the NP:compound oligomer structure, agreeing with the SPR studies which demonstrated that the formation of the NP:compound oligomer does not affect RNA binding affinity. Three residues observed in the ligand binding pocket, Y52, Y289, and N309, have also been observed critical to compound potency (Table 5 above). Y52 and Y289 are observed to play a direct role in compound binding through packing interactions with the nitro-aryl ring. Interestingly, N309 participates in a novel NP:NP interaction and is not directly involved in compound binding. Y52 also contributes to a novel interaction between compound cross-linked NP monomers. These data suggest that binding energetics emanate from two sources, NP:compound interactions and NP:NP interactions, both of which are crucial to compound potency.

The AUC and DLS experiments demonstrated that the size of the NP:compound complex was an octamer but only a hexamer was present in the x-ray structure. The hexamer observed in the structure could represent an intermediate state not observed in the solution based experiments. While trimers are predominant, both monomers and tetramers of NP have been observed in electron micrographs of NP in solution (Ye et al. 2006). In addition, it is unlikely that the trimer represents the physiological relevant RNA-binding unit, as larger oligomers have been observed in electron micrographs of NP bound to RNA. The same mechanism observed in the structure can be extrapolated to the octamer, with compounds either cross-linking the known NP tetramers to form an octamer or the cross-linked NP hexamer recruiting another NP:compound dimer.

While well-defined in vitro, the composition of the NP:compound complex is likely variable in both size and composition in vivo and would be determined by which other macromolecules are also bound to NP. NP's roles in viral replication require interactions with a vRNA and numerous other cellular or viral proteins, including the vRNA polymerase, viral Matrix protein, actin, alpha importins, and the exportin, CRM1. Both the actin and polymerase binding sites have been localized to the body domain of NP (Portela et al., “The influenza virus nucleoprotein: a multifunctional RNA-binding protein pivotal to virus replication”, J. Gen. Virol., 83(Pt. 4):723-734 (2002)). As the body domain comprises the compound binding site, interactions with these proteins may be directly inhibited by the formation of the NP:compound complex. However, the primary mechanism of antiviral activity has been observed to be direct antagonism of nuclear transport of NP and RNPs (unpublished data; (Kao et al. 2010)). Nuclear transport of NP is crucial for viral replication as Influenza is one of the few RNA viruses to replicate in the nucleus. NP has been observed to play critical roles in the nuclear trafficking of RNPs (Boulo et al., “Nuclear traffic of influenza virus proteins and ribonucleoprotein complexes”, Virus Res., 124(1-2):12-21 (2007)). NP has two nuclear localization signals (non-conventional N-terminal NLS M1-M13 (Wang et al., “The NPI-1/NPI-3 (karyopherin alpha) binding site on the influenza a virus nucleoprotein NP is a nonconventional nuclear localization signal”, J. Virol., 71(3):1850-1856 (1997)) and classical bipartite NLS K198-R216 (Weber et al., “A classical bipartite nuclear localization signal on Thogoto and influenza A virus nucleoproteins”, Virology, 250(1):9-18 (1998)) and has been demonstrated to bind to a number of human importins (a1, a3 and a5) both in vitro and in vivo (O'Neill et al., “The influenza virus NEP (NS2 protein) mediates the nuclear export of viral ribonucleoproteins”, EMBO J., 17(1):288-296 (1998)). Neither region of the protein containing the known NLS sequences were modeled in the structure reported here. The lack of observed density for these regions on the protein indicates structural flexibility and as such both NLS signals may remain accessible in the NP:Compound A complex. This suggests that antagonism of nuclear transport is not due to direct masking of NLS sequences, rather the formation of large, insoluble aggregates in vivo.

Taken together our data suggest a complex binding mode, with the compound binding in a 1:1 ratio, inducing oligomerization of NP monomers to form an octamer, with the RNA binding pocket unobstructed. While novel and potent against a laboratory influenza strain (A/WSN 33) this chemotype may prove challenging to develop into a broad spectrum anti-influenza treatment. Commonly circulating Influenza A viruses, like H3N2 (Brisbane/10/2007) and H1N1 (A/SolomonIslands/3/2006) have each three mutations in the compound binding pocket (FIG. 10). While only two of these mutations have been evaluated (N309 and Y52) and observed to be critical, the others may prove requisite for compound binding as well. Kao et al. report similar findings by demonstrating reduced potency against both H3N2 and H5N1 (A/Vietnam/1194/04) viruses. The emergence of the novel swine-origin influenza A H1N1 (A/CA/07/2009) further suggests that this is a region of variability, with 3 unique compound binding site mutations, including Y289 which has been shown necessary for compound binding. The structural and biophysical data presented here should facilitate the rational design of antiviral compounds with broad strain coverage not reliant on interactions with the known variable compound binding site residues.

### Description and Location of the Structure Coordinates Tables

The structure coordinates of 1) H1N1 NP Swine flu (apo) accorded PDB ID: 4DYS; 2) H1N1 NP WSN/A triple mutant (apo) accorded PDB ID: 4DYT; 3) H1N1 NP WSN/A bound to Compound B accorded PDB ID: 3RO5; 4) H1N1 NP WSN/A bound to Compound A accorded PDB ID: 3TG6; 5) H1N1 NP WSN/A bound to compound G accorded PDB ID: 4DYA; 6) H1N1 NP WSN/A bound to Compound F accorded PDB ID: 4DYN; 7) H1N1 NP WSN/A bound to Compound E accorded PDB ID: 4DYB; and 8) H1N1 NP WSN/A bound to Compound D accorded PDB ID: 4DYP were deposited with the RCSB Protein Data Bank (www.pdb.org, Berman, H. M. et al., “The Protein Data Bank”, Nucleic Acids Research, 28:235-242 (2000) and www.wwpdb.org, Berman, H. M. et al., “Announcing the worldwide Protein Data Bank”, Nature Structural Biology, 10(12):98 (2003)).

### Example I

### Antiviral Compounds

The compounds utilized herein have been described in co-pending U.S. provisional applications 61/380,728, filed Sep. 8, 2010 and 61/387,186, filed Sep. 28, 2010. Both provisional applications are herein incorporated by reference in their entirety.

### Example II

### Cloning, Protein Expression, and Purification of NP

An NP construct comprising amino acids 8-498 from A/WSN/33 virus was cloned in pET28A and contained an N-terminal hexahisidine tag. NP was expressed in E. coli using the Rosetta II (DE3) strain (EMD). Transformed cells were cultured in Terrific Broth (Teknova) at 37° C. with shaking until OD₆₀₀=4, at which time the temperature was reduced to 25° C. At OD₆₀₀=6, 0.4 mM IPTG was added and the cells cultured for an additional 3 hours. The cells were pelleted by centrifugation and frozen at −80° C. The frozen cell pellet was thawed in 25 mM Tris pH 7.5, 1M NaCl, 0.2% NP-40, 10% glycerol with 5 units/mL benzonase and 0.1 mg/mL lysozyme. The cells were lysed by sonication and NP was purified using Ni-NTA affinity, SUPERDEX® 200 gel filtration, and heparin SEPHAROSE® chromatographies. The final protein buffer was 25 mM Tris pH 7.5, 1M NaCl, 10% glycerol, 5 mM DTT.

### Example III

### Thermal Stability Enhancement

The ability of compounds to confer increased thermal stability of NP was measured with a THERMOFLUOR® instrument (3-Dimensional Pharmaceuticals) by monitoring the fluorescence enhancement of an external probe (1-anilino-8-naphthalene sulfonate [ANS]). Fluorescence was monitored with a digital camera equipped with a 500±30-nm filter for detection. The unfolding reactions were carried out in a 384-well plate in a 5 μl volume with 1.5 μM NP±2.5 μM-25 μM compound, 100 μM ANS, and 8% (v/v) DMSO in buffer containing 25 mM Tris pH 7.5, 1M NaCl, 10% glycerol, 5 mM DTT. Reactions were incubated at 25° C. for 1 hr prior to thermal scanning and then overlaid with 3 μl of polydimethylsiloxane DC200 oil (Sigma) to prevent evaporation. Unfolding reactions were monitored by ramping the temperature in 1° C. increments from 25 to 80° C. with a 60 s equilibration time followed by four 10 s exposures (plus one dark-field image) per temperature point. An average intensity per well was calculated by integrating pixel intensities per well and averaging the four exposures. The resulting denaturation curves were analyzed with THERMOFLUOR^{SM} Analysis software (3-Dimensional Pharmaceuticals) to determine thermal melting (Tₘ) values (where Tₘ is the temperature at which the reaction was half complete).

### Example IV

### NMR Experiments

Direct binding of compounds to NP was confirmed using NMR spectroscopy with a T₂ relaxation experiment (Redfield 1978). NMR experiments were carried out at 25° C. on a Varian Inova 600 MHz NMR spectrometer operating at a 599.549 MHz ¹H frequency equipped with a Varian 5 mm ¹H/¹³C/¹⁵N triple resonance, triple-axis PFG probe. All samples were approximately 200 μL in a 3 mm NMR tube and consisted of 10-20 μM compound±15 μM NP in 25 mM sodium phosphate, pD 7.1, 1M NaCl, 8% DMSO (d-6), 92% D₂O. 1024-2048 scans were collected per 50 millisecond delay. Dynamic light scattering—The effect of compounds on the oligomerization NP was measured with a Wyatt Dynamic Light scattering plate reader instrument. The reactions were carried out using a 384-well plate with a 20 μl reaction volume containing 1 μM NP±2 μM compound in buffer containing 25 mM Tris pH 7.5, 1M NaCl, 10% glycerol, 5 mM DTT, 8% (v/v) DMSO. All reactions were incubated at 25° C. for 1 hr prior to DLS analysis. The samples were then passed through a 0.2 μm filter (Pall), pipetted into the reaction plate and overlaid with 10 μl of polydimethylsiloxane DC200 oil (Sigma) to prevent evaporation. Each DLS experiment consisted of at least three individual measurements of 20 acquisitions with a 5 second data acquisition time, and was carried out at 25° C. The diffusion coefficients were corrected for the buffer composition by use of the standard conversion equation within Dynamics software version 6 (Wyatt Technologies). Intensity autocorrelation functions were fitted using the Regularization algorithm in the Dynamics software.

### Example V

### Analytical Ultracentrifugation

Samples for sedimentation velocity experiments contained 10 μM NP±10 μM compound in 50 mM sodium phosphate pH=7.4, 1M NaCl, 10% glycerol, 8% DMSO, 5 mM DTT. All reactions were pre-equilibrated for 1 hr prior to loading SV-AUC cells. SV-AUC was performed using two-channel Epon centerpieces with sapphire windows, spinning at 50,000 rpm collecting data at 280 nm, 360 nm and with interference at 20° C. in an XL-I ultracentrifuge (Beckman). Samples were buffer-matched using 50 mM sodium phosphate pH 7.4, 1M NaCl, 10% glycerol, 8% DMSO, 5 mM DTT. The partial specific volume of NP was calculated from amino acid composition to be 0.724 mL/g and the solvent density was estimated from the sum of the density increments of the buffer components to be 1.05 g/mL (both calculations were made using Sednterp V 1.09). Data analysis was performed using DCDT+2.1 (J. Philo, Thousand Oaks, Calif.). Samples for sedimentation equilibrium experiments contained 10 μM NP±10 μM or 20 μM compound in 50 mM sodium phosphate pH=7.4, 1M NaCl, 10% glycerol, 8% DMSO, 5 mM DTT. Sedimentation equilibrium analysis was performed using six-channel Epon centerpieces with sapphire windows spinning at 7,000, 10,000, and 13,000 rpm. Scans were collected at 280 nm and 360 nm and equilibrium was verified using rms vs. time comparisons of the series of scans collected at each speed (Match module within HeteroAnalysis). Equilibrium was typically established within 24 hours of centrifugation. Global nonlinear least squares fits were performed using HeteroAnalysis 1.1.22 (J. Cole, Storrs, Conn.) to fit a sum of exponential equations to the data. SE-AUC measurements were made in either duplicate or triplicate. The extinction coefficient used to determine binding stoichiometry was calculated for NP to be 5.73 e⁺⁴ M⁻¹ cm⁻¹ at 280 nm (using Sednterp V 1.09) and was measured for Compound A to be 5.28 e⁺³ M⁻¹ cm⁻¹ at 360 nm.

### Example VI

### Surface Plasmon Resonance

Biotinylated 2-O-methylated RNA oligonucleotide with the sequence 5′-biotin-UAG AAA CAA GGG UGU UUU UUC AGA UCU AUU AAA CUU CAC CCU GCU UUU GCU-3′ was purchased (Dharmacon) and immobilized on an SA sensor Chip (GE Healthcare), according to the manufacturer's instructions. SPR measurements were collected with a BIACORE® 2000 (GE Healthcare) at 25° C. in 25 mM Tris pH 7.5, 1M NaCl, 10% glycerol, 5 mM DTT, 8% (v/v) DMSO. Varying concentrations of NP (125 nM-0 nM) or NP:compound complex (125 nM-0 nM NP with 500 nM Compound A) were flowed in duplicate over the immobilized RNA at 45 μL/min and referenced to an unmodified surface. Regeneration between injections was completed by a 1 minute pulse of 2M NaCl. Data processing and kinetic analysis were performed using Scrubber software (version 2.0, BioLogic Software, Campbell, Australia). Processed data were double referenced and globally fit to a simple 1:1 interaction model.

### Example VII

### Antiviral Assays

Madin Darby canine kidney (MDCK) cells and bovine kidney (MDBK) cells and influenza A/WSN/33 were obtained from the American Type Culture Collection (ATCC, Manassas, Va.). Influenza A/Solomonlslands, A/Brisbane and A/CA/07/2009 were obtained from the laboratory of Alexander Klimov (Centers for Disease Control, Atlanta, Ga.). Test compounds, at 100× the final test concentration, were serially diluted in DMSO in 3-fold steps. One μl of diluted compound was added to each well of a 96-well plate. Cells were seeded at 4.5×10⁵ cells per ml. Susceptibility of viruses was determined by incubation in the presence of serial dilutions of each compound. Influenza A/WSN/33 and A/WSN/33-N309K were assayed in MDBK cells using E-MEM supplemented with 2% FBS. All other viruses were assayed in MDCK cells using E-MEM supplemented with 0.125% BA and 1 μg/ml TPCK-treated trypsin. All viruses were tested at a multiplicity of infection (MOI) of 0.001 plaque forming units (PFU)/cell 48 hrs post infection, viral replication in the presence of inhibitor was determined by measuring viral neuraminidase (NA) activity (Eichelberger et al. 2008). Influenza NA will activate the quenched substrate 2′-(4-Methylunbelliferyl)-α-D-N-acetylneuraminic acid (MUNANA) resulting in the generation of fluorescence. A 5× substrate solution was added to yield a final concentration of 100 μM MUNANA, 50 mM MES, 2 mM CaCl₂ and 0.25% NP-40. After a 30 minute incubation at 37° C. the plates were read on a fluorescence plate reader set at 360 nm excitation and 460 nm emission.

### Example VIII

### X-ray Crystallization and Structure Determination

A nucleoprotein construct comprising amino acids 8 to 498 was cloned with a Hexa-Histidine tag at the C-terminus in pET30 expression vector. Expression and purification conditions were as described above except the final buffer consisted of 20 mM Tris/HCl pH 7.5, 200 mM NaCl, 10% glycerol, and 5 mM DTT. The protein was concentrated to 9 mg/ml and complexed with compounds (final compound concentration was 2 mM) prior to crystallization. Crystals were observed in 10 mM NaAc pH 4.5, 0.1 M NaCl, 0.1 M NaCltrate pH 5.25, 0.5 M LiCl, and 16% PEG 4000. Crystals were spherical and 150 μm in diameter. Crystals were harvested and cryofrozen using the PROTEROS® Free-Mounting System, in which humidity conditions surrounding the protein crystals were controlled and optimized. Cryo-solution (20% PEG 200) was added to the crystals using the Picodropper during freezing. Data was collected at the Swiss Light Source (beamline PXI/X06SA) and processed using XDS and XSCALA. The structure was solved by molecular replacement using PDB 2IQH structure used as a search model (MOLREP). Refinement was performed using REFMAC with the program MIFit.

What is claimed is:

1. A crystalline form, wherein the form is a co-crystal comprising H1N1 nucleoprotein and an antiviral compound selected from the group consisting of Compound A and analogs thereof.

2. The crystalline form of claim 1, wherein the crystalline form is described by the coordinates deposited as PDB ID: 4DYP.

3. The crystalline form of claim 1, wherein the crystalline form is described by the coordinates deposited as PDB ID: 3RO5.

4. The crystalline form of claim 1, wherein the crystalline form is described by the coordinates deposited as PDB ID: 3TG6.

5. The crystalline form of claim 1, wherein the crystalline form is described by the coordinates deposited as PDB ID: 4DYB.

6. The crystalline form of claim 1, wherein the crystalline form is described by the coordinates deposited as PDB ID: 4DYN.

7. The crystalline form of claim 1, wherein the crystalline form is described by the coordinates deposited as PDB ID: 4DYA.

8. A H1N1 NP antiviral binding site comprising H1N1 NP monomers from different H1N1 NP trimers.

9. The H1N1 NP antiviral binding site of claim 9, wherein the antiviral binding site comprises amino acids S376, E53, R99, S50, Y313, Y52, A284 of monomer B and Y289, Y52, A284, N309, R305 and Y289 of monomer A.

10. The HINT NP antiviral binding site of claim 10, wherein the antiviral binding site comprises amino acids Y52, Y289 and N309.

11. A method of identifying an H1N1 antiviral comprising:
(a) providing the atomic coordinates of H1N1 NP polypeptide defining a three-dimensional structure of H1N1 NP polypeptide;
(b) identifying chemical entities or fragments with the potential to bind to the antiviral binding site of claim 10;
(c) synthesizing or acquiring the test compound; and
(d) determining the ability of the test compound to generate NP oligomers.


SEQUENCE LISTING (9 sequences)

SEQ ID NO: 1 - RNA, 51 nt, Artificial
  Biotinylated 2-O-methylated RNA
uagaaacaaggguguuuuuucagaucuauuaaacuucacccugcuuuugcu

SEQ ID NO: 2 - protein, 49 aa, Influenza A /Brisbane/10/2007
  misc_feature: Viral nucleoprotein (amino acids 51-100)
DHEGRLIQNSLTIEKMVLSAFDERRNKYLEEHPSAGKDPKKTGGPIYRR

SEQ ID NO: 3 - protein, 49 aa, Influenza A/Solomon Islands/3/2006
  MISC_FEATURE: Viral nucleoprotein (amino acids 51-100)
DYEGRLIQNSLTIERMVLSAFDERRNKYLEEHPSAGKDPKKTGGPIYKR

SEQ ID NO: 4 - protein, 49 aa, Influenza A/CA/07/2009
  MISC_FEATURE: Viral nucleoprotein (amino acids 51-100)
DYDGRLIQNSITIERMVLSAFDERRNKYLEEHPSAGKDPKKTGGPIYRR

SEQ ID NO: 5 - protein, 49 aa, Influenza A/WSN/33
  MISC_FEATURE: Viral nucleoprotein (amino acids 51-100)
DYEGRLIQNSLTIERMVLSAFDERRNKYLEEHPSAGKDPKKTGGPIYRR

SEQ ID NO: 6 - protein, 49 aa, Influenza A/Brisbane/10/2007
  MISC_FEATURE: Viral nucleoprotein (amino acids 281-330)
YGPAVSSGYAFEKEGYSLVGIDPFKLLQNSQIYSLIRPNENPAHKSQLV

SEQ ID NO: 7 - protein, 49 aa, Influenza A/Solomon Islands/3/2006
  MISC_FEATURE: Viral Nucleoprotein (amino acids 281-330)
YGPAVASGYDFEKEGYSLVGVDPFKLLQTSQVYSLIRPNENPAHKSQLV

SEQ ID NO: 8 - protein, 49 aa, Influenza A/CA/07/2009
  MISC_FEATURE: Viral nucleoprotein (amino acids 281-330)
YGLAVASGHDFEREGYSLVGIDPFKLLQNSQVVSLMRPNENPAHKSQLV

SEQ ID NO: 9 - protein, 49 aa, Influenza A/WSN/33
  MISC_FEATURE: Viral nucleoprotein (amino acids 281-330)
YGSAVASGYDFEREGYSLVGIDPFRLLQNSQVYSLIRPNENPAHKSQLV